(19) United States
(12) Patent Application Publication
(10) Pub. No.: US 2025/0226067 A1
(43) Pub. Date: Jul. 10, 2025

(54) Codified Tool for Enabling High Value Conveyance of Information

(71) Applicants: Thomas D. Baker, Fishers, IN (US); Patrick W. Thomas, Fishers, IN (US)

(72) Inventors: Thomas D. Baker, Fishers, IN (US); Patrick W. Thomas, Fishers, IN (US)

(21) Appl. No.: 18/406,339

(22) Filed: Jan. 8, 2024

(51) Int. Cl. G16H 10/60; G06F 16/34; G06F 40/106; G06F 40/109; G06F 40/166; G16H 10/20

(52) U.S. Cl. CPC G16H 10/60; G06F 16/345; G06F 40/106; G06F 40/109; G06F 40/166; G16H 10/20

(57) ABSTRACT

Methods and tools are described that provide a reporting functionality that can identify important information and present a report about the important information that highlights important data to the user using the fewest possible words to maximize transfer of information. The described methods and tools are generally described in the field of elder care but are applicable to other personal history reports as well.

### CROSS-REFERENCE TO RELATED APPLICATIONS

Provisional application No. 63/479,157 filed on Jan. 9, 2023.

### PRIOR ART

Publication: US20200074703A1, Patent No.: U.S. Pat. No. 10,467,780 B2, Date: Nov. 5, 2019, SYSTEM AND METHOD FOR DATA.
Publication: US20130321425A1, Patent Application: U.S. Ser. No. 13/789,341 events, Date: Dec. 5, 2013, REPORTING MODULES.
Publication: US20040017400A1, Patent Application: U.S. Ser. No. 10/205,736, Date: Jan. 29, 2004, METHOD FOR PROJECT PLANNING
Publication: U.S. Pat. No. 9,613,086B1, Patent: U.S. Pat. No. 9,613,086 B1, Date: Apr. 4, 2017, GRAPHICAL USER INTERFACE FOR GENERATING AND DISPLAYING DATA VISUALIZATIONS THAT USE RELATIONSHIPS.

### FIELD

The present disclosure generally relates to data capture and visualization of personal history of a host, and in particular ways to present such data.

The present invention is directed to provide a visual aid. More specifically, a visual aid to assist health care professionals in the care of patients and residents. The utility of the invention is enabling connection with patients or customers and is enumerated beyond healthcare, for example rehabilitation centers, hospitality, hospice care, and home health care.

### BACKGROUND OF THE INVENTION

Agitation and/or aggression are estimated to affect up to approximately 80% of patients with dementia, and Alzheimer's disease is the most common form of dementia. Agitation in dementia has a significant negative impact on functional ability, quality of life, caregiver burden, institutionalization, health care expenses, and mortality.

10% of US adults aged 65 or older have dementia, while another 22% have cognitive impairment.¹

1.3 million elderly adults (65 and older) live in nursing homes while an additional 818,000 reside in assisted living facilities. This is important because these seniors are cared for by others, not their family members who have the knowledge of the elderly's life highlights.

Proper health care is a vital means for longevity of life. Some people need more hands-on assistance as they age. This entails assisted living, skilled nursing, long term care, and memory focused organizations that provide the assistance needed for appropriate elder care. Within these organizations are the health care workers that are tasked with assisting patients and residents on a daily basis, many times with medications, dining experiences, personal hygiene, and overall quality of life. These health care workers need to keep track of dozens of patients and residents while attending to the complex routines associated with those individuals. Often an electronic system is located at a central nursing station that houses individual information ranging from medications needed, to dining habits, to personal preferences. At times, information is printed and carried by the health care professional to assist in their memory and planning for the shift work ahead of them.

Electronic systems that house patient or resident information are often patient or resident specific. These systems exist to aid the health care provider in daily care of their patients and residents, for example US20200074703A1. These systems are often located in a centralized location and interacted with on an as needed basis. Typically, the electronic systems are not mobile. Information needed must be physically interacted with at the centralized location. Historical information outside of direct care is included but often minimal.

It is noted that electronic systems often collect information of a personal nature. It is also noted that most, if not all, electronic data capture systems have the ability to generate reports of some nature. These reports are often, if not always, used to convey information to the user. Most, if not all, reports do not adequately utilize design principles to convey information. Most, if not all, reports do not focus on the use of the report, with the focus being on the conveyance of information (see e.g. US20130321425A1; US20040017400A1; U.S. Pat. No. 9,613,086B1).

With respect to the personal preferences of patients and residents, typical forms used are printed in black and white on a grid based format and carried with the health care provider. This process is mostly devoid of individual, specific historical information and relies upon current, daily information to guide patients and residents in daily life tasks. Often with elder care patients and residents, the ability to live in the current day versus past life experiences becomes intertwined. Miscommunications between health care providers and their patients and residents is the outcome, which further results in increased emotional distress for both the health care provider and the patient or resident.

What is therefore needed in view of the above is a method and tool.

What is therefore further needed is a method and tool to collect patient, resident, or customer information codified using specific design principles to convey information in such a way that persons untrained in the method or tool may be unable to use such information (and is therefore protected), and such information can be made available either physically or electronically in the presence of a useful interaction zone with the stated patient, resident, or customer to improve the outcome of the interaction.

What is therefore further needed in view of the above is a report or visual presentation.

What is therefore still further needed is a report or visual presentation that can be generated from a method using design principles of color coding, information graphical location, lines, and other visual codifications.

### SUMMARY OF THE INVENTION

The following is intended to be a brief summary of the invention and is not intended to limit the scope of the invention.

A tool comprised of a method, design principles, and visual presentation or report (typically but not exclusively from an electronic system) used to provide users immediate access to certain personal data.

The tool would be used by health care professionals in the care of their patients or residents, or similar workers in the deployment of the tool in other scenarios such as rehabilitation, memory care, and long-term care. The deployment is not limited solely to health care and has utility in such industries as rehabilitation centers, hospitality, hospice settings, or home care.

The tool consists of four (4) or five (5) quadrants or groups, more or less, depending on the application of the tool.

The tool also includes additional multi segment lines or shapes which partition information or present codified meaning.

The tool utilizes personal data to populate the quadrants in the case of health care deployments, and or other relevant information within each specific deployment otherwise indicated for the codified display of information.

The historical or other data can be collected via personal interviews.

The historical or other data can be collected via computer generated questionnaire.

The historical or other data populates the quadrants or groups and additional segment lines or shapes as needed to affect the visual transference of information.

The historical or other data is coded to indicate historical or other relevance of the patient and resident to the health care provider, or in the case of other deployment contains significant codified meaning within segments or groups for example: whether the relative is still living or is deceased may have different coloring or lines or other codifications.

The tool utilized by a health care provider can facilitate real time discussions with the patient or resident in their care.

The tool utilized in other deployments facilitates the transfer of high value information, codified such that trained personnel are able to utilize the information to achieve specific outcomes related to the care or service of their charge such as maintenance personnel, housekeeping or rehabilitation technicians.

The tool is reproduced physically and presented visually within an actionable location highly functional within the space of interest (in the case of health care deployment might be in a patient room, near their bathroom facility or in a personal space).

Because the tool is presented in a frame (or other embodiment), it can exist on the wall similar to other pictures or artwork or decorations.

### BRIEF DESCRIPTION OF DRAWINGS

The illustration depicts an installation within health care to enumerate design elements and does not limit the plurality of installations or design elements other than those which would inhibit the utility of high value conveyance of information.

FIG. 1. is a design view of the tool showing the overall size (item marked 1), the groups or quadrants (items marked 2), codification using line features (item marked 9), and high value information depicted generically (items marked 9,10)

FIG. 2. is a design view of the tool showing the layout of groups or quadrants (5 groups were chosen in this presentation).

FIG. 3. presents an exemplary block diagram embodiment of a tool generally designated and configured in accordance with the principles of the present invention used in a Long Term Care Facility.

FIG. 4. presents an exemplary block diagram embodiment of a tool generally designated and configured in accordance with the principles of the present invention used in a Memory Care Facility.

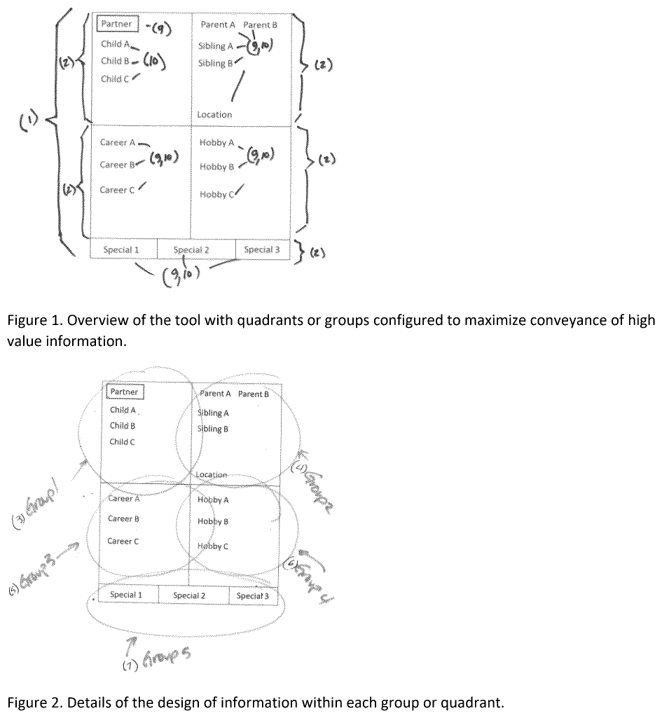

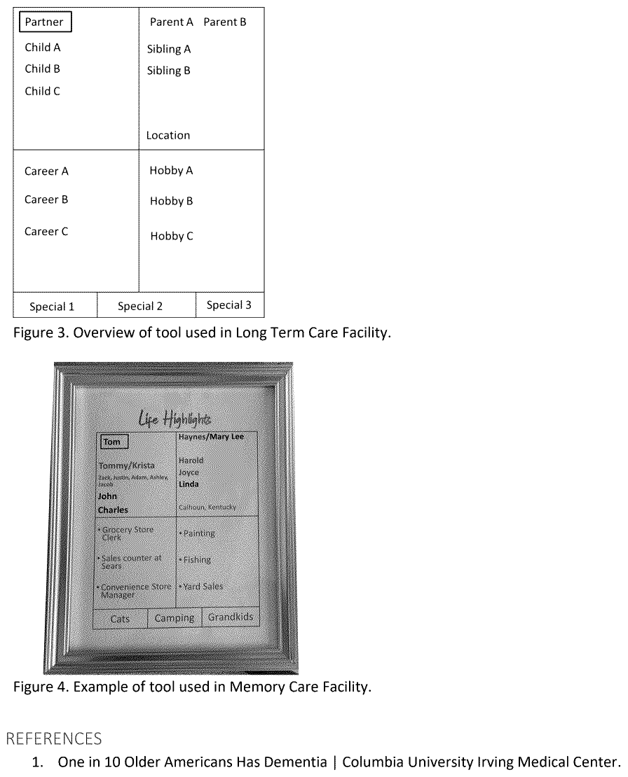

The tool presented as a report or similar, typically printed to standardized sizes such as 8.5″×11″, A4, 11″×17″ or plurality of sizes specific to that deployment and equipment set (FIG. 1, item (1)).

A number of quadrants or groups (such as 3,4,5, or more or less) as needed to codify certain parameters within the plurality of the installation (FIG. 1, item (2)).

Description of the Illustration will continue such that Health Care industry deployment within a Long Term Care (LTC) facility with Memory Care needs. Other deployments within Health Care such as Rehabilitation Facilities, Long Term Care Facilities and similar are contemplated but do not limit the plurality of deployments in such areas which demand or benefit from the codified conveyance of high value information.

Quadrant/(Group 1 in FIG. 2) representing a patient's current life including spouse, partner or significant other, children and other current life persons of high value.

Quadrant/(Group 2 in FIG. 2) representing a patient's childhood life including parents, guardians or caretakers and siblings or significant childhood persons and geographical location of principle life duration.

Quadrant/(Group 3 in FIG. 2) representing a patient's careers, jobs or education experiences.

Quadrant/(Group 4 in FIG. 2) representing a patient's hobbies or interests.

Quadrant/(Group 5 in FIG. 2) representing a patient's high value conversational or special topics.

Topics and words within the tool are codified using design principles such as color coding or other plurality of codification. (FIG. 1, items (9,10))

Tool lines and shapes are codified using design principles such as color coding or other plurality of codification. (FIG. 1, items (9,10))

### DETAILED DESCRIPTION OF THE INVENTION

For the purposes of promoting and understanding of the principles of the invention, reference will not be made to the embodiments illustrated in the drawings and specific language will be used to describe the same. It will nevertheless be understood that no limitation of the scope of the invention is thereby intended, such alterations and further modifications in the illustrated device and such further applications of the principles of the invention as illustrated therein being contemplated as would normally occur to one skilled in the art to which the invention relates. Moreover, the present invention is described herein is described as for the implementation of the tool within a Health Care Facility commonly known as Long Term Care or Memory Care Unit but may be applicable to other Health Care applications and may be applicable to other industries such as rehabilitation centers, hospitality, or hospice settings.

Caregivers in skilled nursing facilities are often understaffed with minimal time to learn the stories of the patients in their care. Typical social histories of patients are captured by social workers and input into various computer systems during intake to which some caregivers have little to no access. The ability to have access to easily consumable, high value personal history information of patients allows caregivers to build stronger relationships and provide a higher quality of life for patients in their care.

The visual tool assembles high value information and presents it in a codified manner, using graphical locations and colors, that reduces triggers for patients while allowing caregivers to quickly, better understand their patient's lives. The visual tool is designed to exist as a static image (or hard copy, or electronic screen) present in the patient's living space. The codified format of the high value information is easily accessible for caregivers as it is always available, requiring no login, no computer access, nor printing of records once deployed. The codified format is designed to be quickly and easily consumed and understood via graphical locations, color definitions, and other codifications.

The visual tool is constructed of graphical locations. Each graphical location represents a specific aspect of a patient's life. Upon review of the visual tool, the user will immediately understand the significance of the information in each graphical location as it pertains to the patient.

Color schemes (and other graphical codifications such as information graphical location, lines or boxes) are also employed to further represent aspects of a patient's life. Color schemes are predetermined so as to allow the user to understand the significance of the color scheme and provide common training among common deployments.

High value information is thus included both graphically, as well as color schemed (and other codifications), to present the highest density of value to the user with a minimal amount of extraneous verbiage.

A minimization of extraneous verbiage, combined with graphical location and color scheme, presents a new and unique way to deliver high value information of patients to the user for ease of use in understanding their patient's life histories.

The tool is a high value transference of concise patient information that, in the hands of a trained user, could be useful in calming, connecting, or conversing.

The tool provides information that can be used to redirect patients who are confused or experiencing an increased level of anxiety.

Further, the tool provides information that enables a more person-centered connection through conversation or other means which provides a happier, calmer environment.

The method of data collection includes discussions or interviews with the patient, or customer, a patient representative, power of attorney or designated contact person with knowledge pertinent to the interview.

The method includes an interview sequence, specific questions presented in a way that does not lead to binary or convergent answers.

The interview or data collection is preferably conducted in person, verbally, by digital data collection form or physical data collection form.

1. A tool comprising high value information in a visual production.

2. A data collection method whereby high value information is collected and distilled to convey parameters in the least number of words.

3. The system of claim 1 above, wherein said tool comprises specific design elements such as color coding, line sizing, line breaking, boxing, font sizes, font selections, typeface, information graphical location, and other common or uncommon formatting as such selected to codify information.

4. The system of claim 2 above, wherein said data collection method comprises specific training that enables distillation and presentation of high value information in the fewest number of words.
